(19) United States
(12) Patent Application Publication
(10) Pub. No.: US 2007/0141082 A1
(43) Pub. Date: Jun. 21, 2007

(54) Cell surface expression vector of parvovirus antigen and microorganisms transformed thereof

(76) Inventors: Moon-Hee Sung, Daejeon (KR); Chul Joong Kim, Daejeon (KR); Chang Min Jung, Seoul (KR); Seung Pyo Hong, Daejeon (KR); Jong Su Lee, Gyeonggi-do (KR); Jae Chul Choi, Daegu (KR); Motomitsu Sewaki, Okayama (JP); Jong Taik Kim, Sungnam (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/588,359

(22) PCT Filed: Feb. 4, 2005
(86) PCT No.: PCT/KR05/00356

§ 371 Date: Aug. 3, 2006

(30) Foreign Application Priority Data
Feb. 4, 2004 (KR) 10-2004-0007321

(51) Int. Cl. A61K 39/155; C12N 15/74; C12N 1/21

(52) U.S. Cl. 424/211.1; 435/472; 435/252.3; 977/802

(57) ABSTRACT

The present invention relates to a surface expression vector expressing a parvovirus antigen on the surface of microorganisms, the vector containing not only a gene encoding the capsid antigen protein of parvovirus causing canine parvovirus (CPV) infection and feline panleukopenia (FLP) but also at least one of pgsB, pgsC and pgsA genes encoding a poly-gamma-glutamate synthetase complex. Also, the present invention relates to microorganisms transformed with the surface expression vector, and parvovirus vaccines containing the transformed microorganisms. According to the present invention, the use of the recombinant bacterial strains expressing the parvovirus antigen on their surface allows the economical production of vaccines for the treatment and prevention of canine parvovirus infection and feline panleukopenia.

### TECHNICAL FIELD

The present invention relates to a vector expressing the capsid antigen protein of a parvovirus causing canine parvovirus (CPV) infection and feline panleukopenia (FLP) on the surface of microorganisms, microorganisms transformed with the vector, and a vaccine for the treatment or prevention of canine parvovirus infection and feline panleukopenia, which contains the transformed microorganisms or extracts thereof. More particularly, the present invention relates to a surface expression vector containing not only a gene encoding the capsid antigen protein of a parvovirus causing canine parvovirus infection and feline panleukopenia but also at least one or two among pgsB, pgsC and pgsA genes encoding a poly-gamma-glutamate synthetase complex which is a surface anchoring motif of microorganisms, as well as microorganisms transformed with the vector and a parvovirus vaccine containing the transformed microorganisms as an effective ingredient.

### BACKGROUND ART

Canine parvovirus (CPV) infection has been the most typical infectious disease for canine diarrhea since its development was reported in the summer of 1978 throughout the world. In puppies, this infectious disease shows hemorrhagic enteritis and sometimes myocarditis, as main symptoms, and has an incidence of 42% and a mortality of 20%. A virus causing this canine parvovirus infection is a canine parvovirus which is a member of the family Parvoviridae, genus Parvovirus, and a kind of feline parvovirus. Generally, canine parvovirus is one of the smallest viruses and has a particle diameter of 18-26 nm. Also, it is a single chain DNA virus having no envelope (Siegl et al., Intervirology, 23:61-73, 1985). The protein of this virus has three kinds of polypeptides. CPV is proliferated only in actively growing cells, shows unclear cell degeneration in cell culture, and forms an intranuclear inclusion body (Cowdry type A), and a specific antigen to CPV can be detected by a fluorescent antibody technique. CPV is antigenically and genetically very similar to mink enteritis virus (MEV) and feline panleukopenia virus (FPLV) (Parrish et al., Arch. Virol., 72:267-78, 1982).

Since parvovirus in dogs showing leukopenia with the symptoms of severe emesis and diarrhea was reported for the first time in 1978 in USA, Canada, Austria, etc., this disease has been spread in all countries of the world from the 1980s. Particularly in the case of group breeding, the parvovirus is likely to have higher positive rate and infectious activity. When canine parvovirus invades non-contaminated areas, it causes enteritis in dogs regardless of their ages due to its strong infectivity, thus resulting in high mortality. Once it is spread, all dogs show antibody positivity, and then, disease development tends to be concentrated on puppies (7-14-week old) where maternal antibodies disappear. Clinical symptoms become severe 5 days after oral infection, and when susceptible dogs are exposed to the virus, 100% of the dogs are infected with the virus, about 75% of the dogs have inapparent infection and 25% may have clinical symptoms with high mortality. As early symptoms, dogs are spiritless, loss of appetite and emesis occur, and diarrhea is observed 24-48 hours after the start of emesis. When such conditions are continued, dehydration, bodyweight decrease, and watery and blood-containing diarrhea are observed. Antibodies in serum can be measured 5 days after oral infection and reach the peak at 7-8 days.

Currently, the diagnosis of canine parvovirus (CPV) infection is made based on main clinical symptoms and confirmed by the detection of the virus and an increase of serum neutralizing antibodies. Canine parvovirus has the blood cell aggregation ability by which the HA activity and HI antibody titer in feces can be measured, thus diagnosing relatively easily. Moreover, the detection of a specific antibody in blood and the confirmation of a characteristic IgM antibody appearing at the early stage of infection become important for diagnosis.

There is no fundamental therapeutic method against CPV, and only conservative therapy can be used. The best method to prevent infection with canine parvovirus and feline panleukopenia virus is the prevention by a vaccine. A vaccine for the prevention of parvovirus, which has been used till now is either a killed vaccine obtained by culturing parvovirus in tissue, and then inactivating highly pathogenic virus with formalin, or attenuated virus obtained by sub-culturing pathogenic virus over dozens of generations in a laboratory.

The tissue culture of canine parvovirus produces a large number of incomplete viruses (empty particles), thus making it very difficult to produce a high-titer vaccine.

The technology of expressing by attaching the desired protein to the cellular surface of microorganisms is referred to as the cell surface display technology. This cell surface display technology is to express a foreign protein on the cellular surface using the surface protein of microorganisms, such as bacteria or yeasts, as a surface anchoring motif, and is used in a wide range of applications, including the production of recombinant live vaccines, the construction and screening of peptide/antibody libraries, whole cell absorbents and bioconversion catalysts. The application range of this technology is determined depending on what protein is expressed on the cell surface, thus, the industrial application potentiality of the cell surface display technology can be said to be significant.

For successful cell surface display, a surface anchoring motif is most important.

How effectively a motif capable of expressing a foreign protein on the cell surface is selected and developed is the core of this technology. Accordingly, a surface anchoring motif with the following properties should be selected. First, it should have a secretory signal helping the foreign protein to pass through the inner cell membrane, and to reach to the cell surface. Second, it should have a target signal helping the foreign protein to be stably attached to the outer cell membrane surface. Third, it should be expressed on the cell surface at large amounts but has little or no effect on the growth of cells. Fourth, it should be stably expressed regardless of the protein size, without causing a change in the three-dimensional structure of the foreign protein. However, a surface anchoring motif meeting all the above requirements was not yet been developed.

Cell surface anchoring motifs, which have been known and used till now, are broadly classified into four kinds, i.e., outer membrane proteins, lipoproteins, secretory proteins, and surface organ proteins such as flagella proteins. In the case of gram-negative bacteria, proteins present on the outer cell membrane, such as LamB, PhoE (Charbit et al., J. Immunol., 139:1658, 1987; Agterberg et al., Vaccine, 8:85, 1990) and OmpA, were mainly used. Moreover, lipoproteins, such as TraT (Felici et al., J. Mol. Biol., 222:301, 1991), PAL (peptidoglycan associated lipoprotein) (Fuchs et al., Bio/Technology, 9:1369, 1991) and Lpp (Francisco et al., Proc. Natl. Acad. Sci. USA, 489:2713, 1992), were also used. Furthermore, the expression of foreign proteins was also attempted using FimA, a fimbriae protein such as the FimH adhesion of type 1 fimbriae (Hedegaard et al., Gene, 85:115, 1989), or a pili protein such as a PapA pilu subunit as surface anchoring motifs. In addition, there are reports that an ice nucleation protein (Jung et al., Nat. Biotechnol., 16:576, 1998; Jung et al., Enzyme Microb. Technol., 22:348, 1998; Lee et al., Nat. Biotechnol., 18:645, 2000), the pullulanase of Klebsiela oxytoca (Komacker et al., Mol. Microl., 4:1101, 1990), the IgA protease of Neiseria (Klauser et al., EMBO J., 9:1991, 1990), E. coli adhesion AIDA-1, the VirG protein of shigella, a fusion protein of Lpp and OmpA, can be used as surface anchoring motifs. In the case of the use of Gram-positive bacteria, there is a report that a malaria antigen was effectively expressed using Staphylococcus aureus-derived protein A and an FnBPB protein, as surface anchoring motifs. In addition, there are reports that the surface coat protein of lactic acid bacteria was used in surface expression and that a Streptococcus pyogenes-derived M6 protein (Medaglini, D et al., Proc. Natl. Acad. Sci. USA., 92:6868, 1995), the S-layer protein EA1 of Bacillus anthracis, and the surface protein of Gram-positive bacteria such as Bacillus subtilis CotB, etc., were used as surface anchoring motifs.

The present inventors already developed a novel vector effectively expressing a foreign protein on the surface of microorganisms using pgsBCA genes encoding a Bacillus sp. strain-derived poly-gamma-glutamate synthetase complex as new surface anchoring motifs, as well as a method for expressing a large amount of foreign protein on the surface of microorganisms transformed with the vector (WO 03/14360). Many studies were performed in an attempt to stably express the antigen or epitope of pathogenic organisms in bacterias where mass production is possible by genetic engineering techniques using the above-described surface anchoring motifs. It was reported that, particularly when a foreign immunogen expressed on the surface of non-pathogenic bacteria is orally administered alive, a more lasting and strong immune response than that of the prior vaccine using the prior attenuated pathogenic bacteria or viruses can be induced. This induction of the immune reaction is known to be because the surface structures of the bacteria act as adjuvants increasing the antigenicity of the surface-expressed foreign protein, and an in vivo immune response to the live bacteria occurs. The development of a recombinant live vaccine of non-pathogenic bacteria using this surface expression system is noticeable.

### DISCLOSURE OF INVENTION

Accordingly, the present inventors have found that a large amount of a parvovirus antigen selected by the gene and protein analysis can be expressed on the surface of non-pathogenic microorganisms with food safety guaranteed, such as lactic acid bacteria, using pgsBCA genes encoding a Bacillus sp. strain-derived poly-gamma-glutamate synthetase complex, as surface anchoring motifs, and developed a more economical, stable, preventive vaccine inducing the production of a parvovirus antibody in blood of the living body and mucosal immunization by administering these microorganisms to mice orally or rhinally.

It is an object of the present invention to provide a vector capable of expressing a parvovirus antigen using the surface expression system of microorganisms, and microorganisms transformed with the vector.

Another object of the present invention is to provide transformed microorganisms having a parvovirus antigen expressed on their surface, and a vaccine for the prevention of parvovirus, which contains either a parvovirus antigen extracted from the transformed microorganisms or a parvovirus antigen purified from the microorganisms, as an effective ingredient.

To achieve the above objects, in one aspect, the present invention provides a surface expression vector comprising at least one selected from the group consisting of pgsB, pgsC and pgsA genes encoding a poly-gamma-glutamate synthetase complex, and a gene encoding a parvovirus capsid antigen protein selected from the group consisting of VP2-1, VP2-2 and VP2.

In the present invention, the surface expression vector is preferably pHCE2LB:pgsA:VP2-1, pHCE2LB:pgsA:VP2-2 or pHCE2LB:pgsA:VP2.

Any microorganisms may be used in the present invention if they have no toxicity upon in vivo application or have been attenuated. Preferably, as gram-negative bacteria, E. coli., Salmonella typhi, Salmonella Typhimurium, Vibrio cholerae, Mycobacterium bovis and shigella, and as gram-positive bacteria, Bacillus, Lactobacillus, Lactococcus, Staphylococcus, Listeria monocytogenes and Streptococcus, may be suitably selected. Particularly, edible microorganisms, such as lactic acid bacteria, are preferably selected.

In another aspect, the present invention provides a method for preparing a parvovirus capsid antigen protein, comprising culturing the transformed microorganisms to express the parvovirus capsid antigen protein on the surface of the microorganisms.

In still another aspect, the present invention provides a vaccine for the prevention of parvovirus, the vaccine containing the antigen protein prepared by said method, as an effective ingredient. In the present invention, it is possible to use microorganisms themselves having the antigen protein expressed on their surface, a crude extract from cell membrane components obtained by destroying the microorganisms, or an antigen protein purified from the microorganisms.

In yet another aspect, the present invention provides a method for preparing a parvovirus capsid antigen protein, comprising culturing the transformed lactic acid bacteria to express the parvovirus capsid antigen protein on the surface of the lactic acid bacteria.

In still another aspect, the present invention provides a lactic acid bacteria produced by said method, which have a parvovirus capsid antigen protein expressed on its surface.

In further another aspect, the present invention provides a vaccine for the prevention of parvovirus, the vaccine containing said lactic acid bacteria, a capsid antigen protein extracted from the lactic acid bacteria, or a capsid antigen protein purified from the lactic acid bacteria, as an effective ingredient.

The vaccine according to the present invention can be used as a medical drug for the prevention of parvovirus infection caused by parvovirus. The inventive vaccine can be administered orally, ingested as food, injected subcutaneously or celiacly, or administered rhinaly.

In still another aspect, the present invention provides a feedstuff additive or preparation for the prevention of parvovirus, which contains said microorganisms or a parvovirus capsid antigen protein obtained by culturing the microorganisms, as an effective ingredient.

Since infection with a parvovirus causing canine parvovirus and feline panleukopenia is known to be infected mainly by an oral route, the infection is inferred to occur on the mucosal surface of a digestive organ. Thus, the prevention of infection by mucosal immunization is very important. Accordingly, the microorganisms having the parvovirus antigen expressed on their surface have an advantage in that they can more effectively induce the formation of an antibody on a mucosa (mucosal response), thus, it is expected that an orally or rhinally administered vaccine using the transformed microorganisms themselves will be more effective for the defense of parvovirus than a parenteral vaccine.

### BRIEF DESCRIPTION OF DRAWINGS

FIG. 1 shows the results of analysis for the relation between the antigenic site of parvovirus and the capsid protein VP2 of parvovirus by a hydrophilicity plot, the Kyte-Doolittle method, an antigen index, the Jameson-wolf method, and a surface probability plot, the Emini method.

FIG. 2(A) is a gene map of the inventive surface expression vector pHCE2LB:pgsA:VP2-1 containing the inventive gram-negative and positive microorganisms as a host; FIG. 2(B) is a gene map of the inventive surface expression vector pHCE2LB:pgsA:VP2-2, and FIG. 2(C) is a gene map of the inventive surface expression vector pHCE2LB:pgsA:VP2.

FIG. 3 is a photograph showing the results of Western blot analysis for the protein expression patterns of pgsA-fused capsid proteins VP2-1 and VP2-2 in lactic acid bacteria transformed with each of the inventive surface expression vectors pHCE2LB:pgsA:VP2-1 and pHCE2LB:pgsA:VP2-2. Lane 1 represents the whole cell of Lactobacillus casei, which is a non-transformed host cell, lane 2 represents pHCE2LB:pgsA:VP2-1/Lactobacillus casei, and lane 3 represents pHCE2LB:pgsA:VP2-2/Lactobacillus casei.

FIG. 4 graphically shows the results of enzyme-linked immunosorbent assay (ELISA) for IgG antibody titers against CPV VP2-1 and CPV VP2-2 antigens in the serum of mice orally and rhinally administered with a Lactobacillus casei strain which has been transformed with each of the inventive surface expression vector pHCE2LB:pgsA:VP2-1 and pHCE2LB:pgsA:VP2-2 and confirmed to have an epitope expressed on its surface. “A” represents IgA antibody titer in the oral administration group, and “B” represents IgA antibody titer in the rhinal administration group.

FIG. 5 graphically shows the results of enzyme-linked immunosorbent assay (ELISA) for IgA antibody titers against CPV VP2-1 and CPV VP2-2 antigens in the intestines, bronchia and bronchoalveolar lavage fluids of mice orally and rhinally administered with a Lactobacillus casei strain which has been transformed with each of the inventive surface expression vectors pHCE2LB:pgsA:VP2-1 and pHCE2LB:pgsA:VP2-2 and confirmed to have an epitope expressed on its surface. “A” represents IgA antibody titer in the oral administration group, and “B” represents IgA antibody titer in the rhinal administration group.

### DETAILED DESCRIPTION OF THE INVENTION

Hereinafter, the present invention will be described in more detail by the following examples. However, it will be obvious to a person skilled in the art that these examples are given to provide a better understanding of the present invention and are not construed to limit the scope of the present invention.

Although the antigenic site gene and whole gene of the parvovirus capsid protein were used in the following examples, any antigenic protein genes may also be used alone or in a combination of two or more.

Furthermore, although the outer membrane genes pgsBCA involved in the synthesis of poly-gamma-glutamate, which have been obtained from Bacillus subtilis var. chungkookjang (KCTC 0697BP), were used in the following examples, either vectors prepared with pgsBCA genes obtained from all Bacillus sp. strains producing poly-gamma-glutamate, or microorganisms transformed with these vectors, will also be within the scope of the present invention. For example, either the preparation of vaccine vectors using other strain-derived pgsBCA genes having a homology of at least 80% with the base sequence of pgsBCA genes present in Bacillus subtilis var. chungkookjang, or the use thereof, will also be within the scope of the present invention.

Moreover, although a surface expression vector was prepared only with a pgsA gene among pgsBCA genes in the following examples, the construction of vaccine vectors with all or parts of the pgsBCA genes will also be within the scope of the present invention.

Furthermore, although only Lactobacillus bacteria which is gram-positive bacteria, were used as hosts for the vectors in the following examples, it will also be obvious to a person skilled in the art that when gram-negative or gram-positive bacteria other than such bacteria, transform by the inventive method, the same results can be obtained.

Also in the following examples, only the case where microorganisms themselves transformed with the vaccine vectors were applied as live vaccines to the living body is presented. However, in view of the knowledge in the field of vaccine-related technology, it is to be understood that, even when either a crude extract from said microorganisms (parvovirus antigen protein) or an expressed protein purified from said microorganisms is applied to the living body, the same results can be obtained.

### EXAMPLE 1

### Selection of Antigenic Site Gene in Capsid Antigen Protein VP2 of Canine Parvovirus

The capsid antigen protein VP2 of canine parvovirus is a glycoprotein consisting of 586 amino acids. In the case of canine parvovirus on which many studies have been made, capsid antigen protein VP2 has been mainly studied as a target antigen of a vaccine for inducing and preventing virus infection.

Accordingly, a more effective antigenic fragment was selected by the protein analysis and structural analysis of the capsid antigen protein VP2 of canine parvovirus.

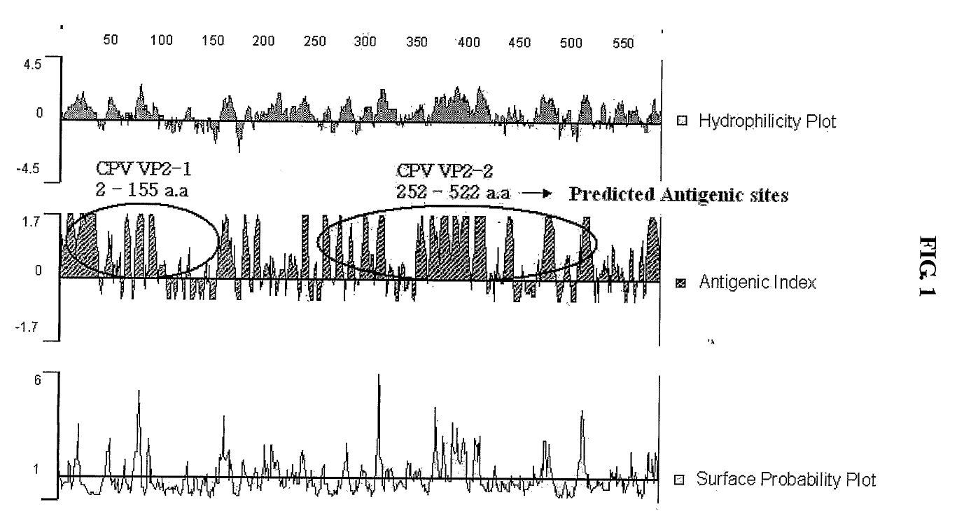

Specifically, the proteins of the antigenic fragment of canine parvovirus capsid antigen protein VP2 were analyzed by a hydrophilicity plot, which is the Kyte-Doolittle method, an antigenic index, which is the Jameson-wolf method, and a surface probability plot, which is the Emini method, and then, VP2-1 and VP2-2 of the whole capsid antigen protein VP2 of canine parvovirus were selected (FIG. 1).

VP2-1 having an amino acid length of 153 (2^{nd}-153^{rd} amino acids) was named “CPV VP2-1”, and a fragment having an amino acid length of 270 (252^{nd}-522^{nd} amino acids) was named “CPV VP2-2.

### EXAMPLE 2

### Construction of Surface Expression Vector pHCE2LB:pgsA:VP2-1

Using pgsA among outer membrane protein genes pgsBCA involved in the synthesis of poly-gamma-glutamate derived from Bacillus sp. strains, vector pHCE2LB:pgsA:VP2-1 capable of expressing the antigenic fragment VP2-1 of canine parvovirus capsid antigen protein VP2 on the surface of gram-negative and gram-positive microorganisms as hosts was constructed.

First, the diarrhea feces of dogs suspected of having canine parvovirus infection in a domestic veterinary hospital was collected, from which virus was isolated. Then, from the isolated virus, virus DNA was extracted. In order to introduce a gene encoding the VP2-1 of CPV into a transformation vector for surface expression (KCTC 10349BP of Human papilloma virus antigen L1), which uses gram-negative and gram-positive microorganisms as hosts and includes an HCE promoter which is a constant high expression promoter in gram-negative and gram-positive general purpose vector pAT, pgsA among outer membrane protein genes (pgsBCA) involved in the synthesis of poly-gamma-glutamate, and human papilloma virus antigen L1 (HPV L1), PCR was performed using a parvovirus gene isolated from dogs as a template, and oligonucleotide having base sequences of SEQ ID NO: 1 (5′-cgc gga tcc agt gat gga gca gtt caa-3′) and SEQ ID NO: 2 (5′-ccc aag ctt aag ctt aaa cat taa aaa ttt ctt-3′) derived from a gene encoding the CPV VP2-1 as primers. As a result, the size of the PCR amplified gene fragment was 459 bp.

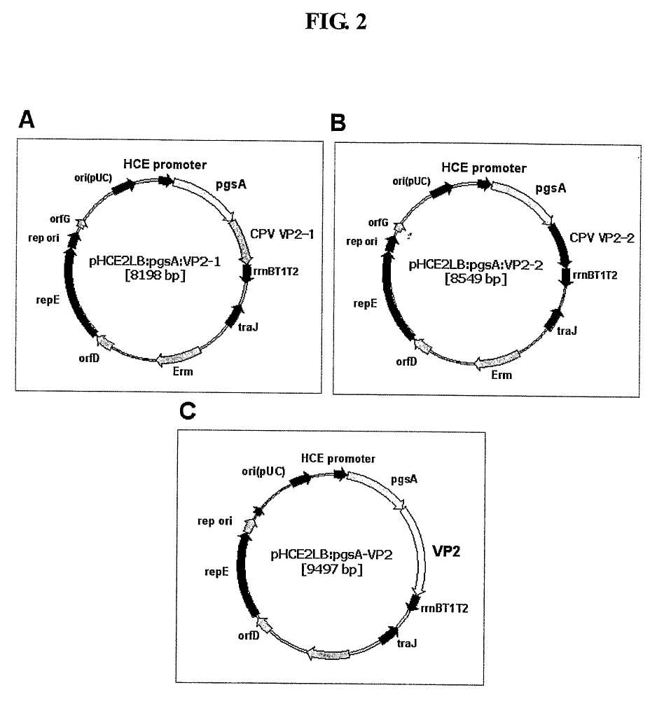

The primers of SEQ ID NO: 1 and SEQ ID NO: 2 were constructed to have recognition sites for restriction enzymes BamHI and KpnI present in surface expression vector pHCE2LB:pgsA obtained by cutting surface expression vector pHCE2LB:pgsA-HPVL1 (KCTC 10349BP) with BamHI and KpnI to remove the HPVL1 gene. The amplified CPV VP 2-1 antigen gene was linked in accordance with decoding codon to the C-terminal end of the outer membrane protein gene pgsA involved in poly-gamma-glutamate synthesis, of the surface expression vector pHCE2LB:pgsA prepared by cutting with restriction enzymes BamHI and KpnI, thus constructing transformation vector pHCE2LB:pgsA:VP2-1 (see FIG. 2(A)).

Gram-positive Lactobacillus bacteria, were transformed with the constructed surface expression vector pHCE2LB:pgsA:VP2-1, and then, the presence of the pHCE2LB:pgsA:VP2-1 plasmid in Lactobacillus bacteria was confirmed.

### EXAMPLE 3

### Construction of Surface Expression Vector pHCE2LB:pgsA:VP2-2

Using pgsA among the Bacillus sp. strain-derived outer membrane protein genes pgsBCA involved in the synthesis of poly-gamma-glutamate, surface expression vector pHCE2LB:pgsA:VP2-2 which can express the antigenic fragment VP2-2 of canine parvovirus capsid antigen protein VP2 on the surface of gram-negative and gram-positive microorganisms as hosts was constructed.

First, in order to introduce the antigenic fragment VP2-2 of canine parvovirus capsid antigen protein VP2, the surface expression vector pHCE2LB:pgsA:VP2-1 constructed in Example 1 was cut with BamHI and KpnI to remove the VP2-1 gene, thus preparing surface expression vector pHCE2LB:pgsA.

In order to introduce a gene encoding the VP2-2 of CPV, PCR was performed using a canine parvovirus gene isolated from dogs as a template, and oligonucleotide having base sequences of SEQ ID NO: 3 (5′-cgc gga tcc cca gta cac tta cta aga-3′) and SEQ ID NO: 4 (5′-ccc aag ctt ggt acc tta aat tct tga cat att-3′), derived from a gene encoding the CPV VP2-2 as primers. As a result, the size of the PCR amplified gene fragment was 810 bp.

The amplified CPV VP2-2 antigen gene was linked in accordance with decoding codon to the C-terminal end of the outer membrane protein gene pgsA involved in the synthesis of poly-gamma-glutamate, of the surface expression vector pHCE2LB:pgsA prepared by cutting with restriction enzymes BamHI and KpnI, thus constructing transformation vector pHCE2LB:pgsA:VP2-2 (see FIG. 2(B)).

Lactobacillus bacteria, which are gram-positive bacteria, were transformed with the constructed surface expression vector pHCE2LB:pgsA:VP2-2, and then, the presence of the pHCE2LB:pgsA:VP2-2 plasmid in Lactobacillus bacteria was confirmed.

### EXAMPLE 4

### Construction of Surface Expression Vector pHCE2LB:pgsA-VP2

Using pgsA of Bacillus sp. strain-derived outer membrane protein genes (pgsBCA) involved in the synthesis of poly-gamma-glutamate, surface expression vector pHCE2LB:pgsA-VP2 which can express the whole of canine parvovirus capsid antigen protein VP2 on the surface of gram-negative and gram-positive microorganisms as hosts was constructed.

First, in order to introduce canine parvovirus capsid antigen protein VP2, the surface expression vector pHCE2LB:pgsA:VP2-1 constructed in Example 1 was cut with BamHI and KpnI to remove the VP2-1 gene, thus preparing surface expression vector pHCE2LB:pgsA.

In order to introduce a gene encoding the VP2 of CPV, PCR was performed using a canine parvovirus gene isolated from dogs as a template, and oligonucleotide having base sequences of SEQ ID NO: 1 (5′-cgc gga tcc agt gat gga gca gtt caa-3′) and SEQ ID NO: 4 (5′-ccc aag ctt ggt acc tta aat tct tga cat att-3′), which encode the CPV VP2 as primers. As a result, the size of the amplified gene fragment was 1563 bp.

The amplified CPV VP2 antigen gene was linked in accordance with decoding codon to the C-terminal end of the outer membrane protein gene pgsA involved in the synthesis of poly-gamma-glutamate, of the surface expression vector pHCE2LB:pgsA prepared by cuffing with restriction enzymes BamHI and KpnI, thus constructing transformation vector pHCE2LB:pgsA-VP2 (see FIG. 2(C)).

E. coli bacteria were transformed with the surface expression vector, and the E. coli strain containing pHCE2LB:pgsA-VP2 was deposited under the accession number KCTC 10590BP on Jan. 31, 2004 with the Korean Collection for Type Cultures (KCTC), Korean Research Institute of Bioscience and Biotechnology (KRIBB), 52 Oun-dong, Yusong-ku, Taejon, Republic of Korea.

Lactobacillus bacteria, which are gram-positive bacteria, were transformed with the constructed surface expression vector pHCE2LB:pgsA-VP2, and then, the presence of the pHCE2LB:pgsA-VP2 plasmid in Lactobacillus was confirmed.

### EXAMPLE 5

### Surface Expression of pgsA-Fused CPV VP2-1 and CPV VP2-2

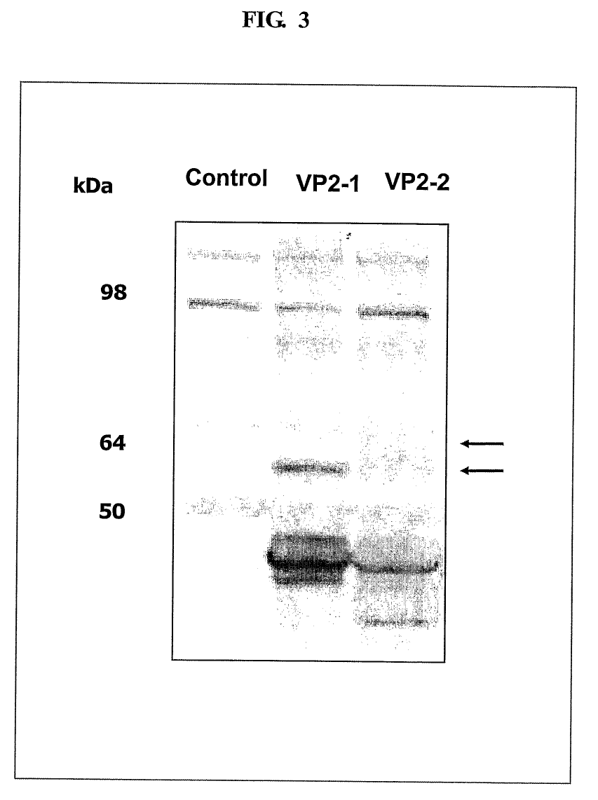

Lactobacillus bacteria transformed with each of the surface expression vectors pHCE2LB:pgsA:VP2-1 and pHCE2LB:pgsA:VP2-2 were cultured, and the expressions of each of pgsA-fused CPV VP2-1 and CPV VP2-2 antigen proteins were examined (see FIG. 3). The bacterial expression of the CPV VP2-1 antigen fused with the C-terminal end of the gene pgsA involved in the synthesis of poly-gamma-glutamate was confirmed by SDS-polyacrylamide gel electrophoresis and Western immunoblotting using a pgsA-specific antibody.

Specifically, a Lactobacillus casei strain transformed with each of pHCE2LB:pgsA:VP2-1 and pHCE2LB:pgsA:VP2-2 was proliferated by stationary culture in MRS medium (Lactobacillus MRS, Becton Dickinson and Company Sparks, USA) at 37° C., thus inducing the surface expression.

The expression-induced Lactobacillus casei strain was denatured with a protein obtained at the same cell concentration so as to prepare a sample. The sample was analyzed by SDS-polyacrylamide gel electrophoresis, and then, the protein fractions were moved to PVDF (polyvinylidene-difluoride) membranes (Bio-Rad). The PVDF membranes to which the protein fractions have been moved were blocked by shaking for 1 hour in a blocking buffer (50 mM Tris HCl, 5% skim milk, pH 8.0), and then, reacted for 12 hours with one thousand fold dilution of rabbit-derived monoclonal anti-pgsA primary antibodies in a blocking buffer. After completion of the reaction, the membranes were washed with buffer and reacted for 4 hours with one thousand fold dilution of biotin-conjugated anti-rabbit secondary antibodies in a blocking buffer. After completion of the reaction, the membranes were washed with buffer and reacted with an avidin-biotin reagent for 1 hour, followed by another washing. The washed membranes were color-developed by the addition of a matrix and a solution of H₂O₂ and DAB as a color development reagent, and analyzed for the specific binding between the HPV L1-specific antibody and the fusion protein (see FIG. 3). In FIG. 3, lane 1 represents the whole cell of Lactobacillus casei, a non-transformed host cell, lane 2 represents transformed pHCE2LB:pgsA:VP2-1/Lactobacillus casei, and lane 3 represents transformed pHCE2LB:pgsA:VP2-2/Lactobacillus casei.

As shown in FIG. 3, fusion protein bands of about 58.6 kDa and about 71.7 kDa by a pHCE2LB:pgsA:VP2-1 plasmid and a pHCE2LB:pgsA:VP2-2 plasmid, respectively, could be confirmed. It can be found that the band of about 59 kDa is a fusion protein of pgsA and CPV VP2-1 because pgsA has the size of about 41.8 kDa and the CPV VP2-1 protein has the size of about 16.8 kDa. Also, it can be found that the band of about 71.7 kDa is a fusion protein of pgsA and CPV VP2-2 because the CPV VP2-2 has the size of about 30 kDa.

### EXAMPLE 6

### Analysis of Vaccine Effect of Lactic Acid Bacteria Having Canine Parvovirus Capsid Antigen Protein Expressed on Surface

Gram-positive bacteria, Lactobacillus casei, were transformed with each of the surface expression vectors pHCE2LB:pgsA:VP2-1 and pHCE2LB:pgsA:VP2-2 constructed in Examples 3 and 4, and the antigen was expressed on the surface of the transformed Lactobacillus casei. Then, the antigenicity of the canine parvovirus capsid antigen protein fused with the outer membrane protein pgsA involved in the synthesis of poly-gamma-glutamate was examined using a mouse model.

Experiments were conducted on four animal groups consisting of a group orally administered with a mixture of lactic acid bacteria expressing CPV VP2-1 and lactic acid expressing CPV VP2-2, a group rhinally administered with a mixture of lactic acid bacteria expressing CPV VP2-1 and lactic acid bacteria expressing CPV VP2-2, and two control groups administered with lactic acid bacteria expressing no antigen. Each animal group consisted of ten 4-6-week old C57BL/6 mice.

Specifically, each of the surface expression vectors pHCE2LB:pgsA:VP2-1 and pHCE2LB:pgsA:VP2-2 according to the present invention was transformed into Lactobacillus casei so as to collect cells with the same bacterial concentration.

The collected cells were washed several times with PBS buffer (pH7.4), and the Lactobacillus bacteria (5×10⁹ cells) having the antigen expressed on their surface were orally administered to 4-6-week old C57BL/6 mice five times at an interval of one-day, and after one week, five times at an interval of one-day, and after 2 weeks, five times at an interval of one-day. Also, the Lactobacillus bacteria (1×10⁹ cells) having the antigen expressed on their surface were rhinally administered to mice three times at an interval of one-day, and after one week, three times at an interval of one-day, and after 2 weeks, three times at an interval of one-day.

At an interval of two-weeks after each of the oral administration and the rhinal administration, the mouse sera were collected and measured for IgG antibody titer against the capsid antigen protein in serum by ELISA, and the mouse intestines were collected and measured for IgA antibody titers against the capsid antigen protein in an intestinal lavage fluid and a bronchoalveolar lavage fluid by ELISA.

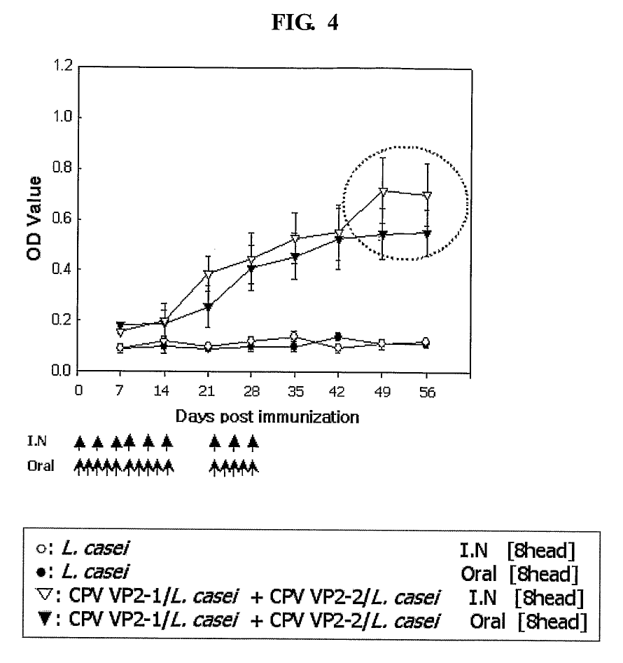

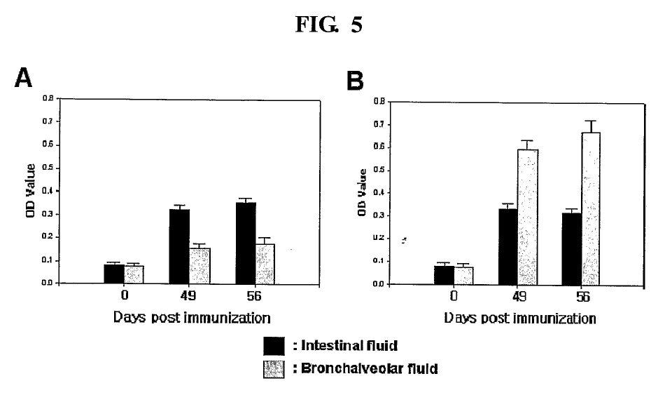

As a result, in the serum, intestinal lavage fluid and bronchoalveolar lavage fluid of the C57BL/6 mice to which the Lactobacillus bacteria transformed with each of pHCE2LB:pgsA:VP2-1 and pHCE2LB:pgsA:VP2-2 have been administered alone or in a mixture, the IgG and IgA antibody titers against the epitopes of canine parvovirus capsid antigen proteins VP2-1 and VP2-2 were significantly higher than those in the control groups (see FIG. 4 and FIG. 5).

Accordingly, it could be found that the inventive microorganisms having the epitopes of canine parvovirus capsid antigen proteins VP2-1 and VP2-2 expressed on their surface would effectively act as mucosa vaccines for oral administration.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof. Those skilled in the art will appreciate that simple modifications, variations and additions to the present invention are possible, without departing from the scope and spirit of the invention as disclosed in the accompanying claims.

### INDUSTRIAL APPLICABILITY

As described above in detail, the inventive transformed microorganisms expressing the parvovirus antigen protein on their surface, and the antigen protein extracted and purified from the microorganisms, can be used as a vaccine for the prevention of parvovirus. Particularly, the inventive recombinant bacterial strains expressing the parvovirus antigen allow producing mucosa vaccines for oral and rhinal administration economically.

1. A surface expression vector comprising at least one gene selected from the group consisting of pgsB, pgsC and pgsA genes encoding a poly-gamma-glutamate synthetase complex, and a gene encoding a parvovirus capsid antigen protein selected from the group consisting of VP2-1, VP2-2 and VP2.

2. The expression vector according to claim 1, wherein the vector is pHCE2LB:pgsA:CVP2-1, pHCE2LB:pgsA:VP2-2 or pHCE2LB:pgsA:VP2.

3. A microorganism transformed with the expression vector of claim 1.

4. The transformed microorganism according to claim 3, wherein the microorganism is selected from the group consisting of E. coli, Salmonella typhi, Salmonella Typhimurium, Vibrio cholerae, Mycobacterium bovis, shigella, Bacillus, lactic acid bacteria, Staphylococcus, Listeria monocytogenes and Streptococcus.

5. The transformed microorganism according to claim 4, wherein the microorganism is lactic acid bacteria.

6. A method for preparing a parvovirus capsid antigen protein, wherein the method comprises the steps of culturing the transformed microorganisms of claim 3, and then expressing the parvovirus capsid antigen protein on the surface of the microorganisms.

7. A vaccine for the prevention of parvovirus, containing the capsid antigen protein prepared by the method of claim 6 as an effective ingredient.

8. The vaccine according to claim 7, wherein the antigen protein is an expressed form on the surface of the microorganism, a crudely extracted form, or a purified form.

9. The vaccine according to claim 7, wherein the vaccine is administered orally or ingested as food.

10. The vaccine according to claim 7, wherein the vaccine is for hypodermic or celiac injection.

11. The vaccine according to claim 7, wherein the vaccine is for rhinal administration.

12. The vaccine according to claim 7, wherein the vaccine is used for the prevention of canine parvovirus infection and feline panleukopenia.

13. A method for preparing a parvovirus capsid antigen protein, wherein the method comprises the steps of culturing the transformed lactic acid bacteria of claim 5, and then expressing the parvovirus capsid antigen protein on the surface of the lactic acid bacteria.

14. A lactic acid bacteria produced by the method of claim 13, comprising a parvovirus capsid antigen protein expressed on their surface.

15. A vaccine for the prevention of parvovirus, containing the lactic acid bacteria of claim 14, a capsid antigen protein extracted from the lactic acid bacteria, or a capsid antigen protein purified from the lactic acid bacteria, as an effective ingredient.

16. The vaccine according to claim 15, wherein the vaccine is administered orally or ingested as food.

17. The vaccine according to claim 15, wherein the vaccine is used for the prevention of canine parvovirus infection and feline panleukopenia.

18. A feedstuff additive for the prevention of parvovirus containing the microorganism of claim 3 or a parvovirus capsid antigen protein obtained by culturing the microorganisms, as an effective ingredient.

19. A feedstuff additive for the prevention of parvovirus containing the lactic acid bacteria of claim 14 or a parvovirus capsid antigen protein obtained by culturing the lactic acid bacteria, as an effective ingredient.

20. A preparation for the prevention of parvovirus containing the microorganism of claim 3 or a parvovirus capsid antigen protein obtained by culturing the microorganisms, as an effective ingredient.


SEQUENCE LISTING (8 sequences)

SEQ ID NO: 1 - DNA, 27 bp, Artificial
  primer
cgcggatccagtgatggagcagttcaa

SEQ ID NO: 2 - DNA, 33 bp, Artificial
  primer
cccaagcttaagcttaaacattaaaaatttctt

SEQ ID NO: 3 - DNA, 27 bp, Artificial
  primer
cgcggatccccagtacacttactaaga

SEQ ID NO: 4 - DNA, 33 bp, Artificial
  primer
cccaagcttggtaccttaaattcttgacatatt

SEQ ID NO: 5 - DNA, 25 bp, Artificial
  primer
cgggatccgccaaccagggacaacg

SEQ ID NO: 6 - DNA, 28 bp, Artificial
  primer
cccaagcttttatggattcattattagc

SEQ ID NO: 7 - DNA, 27 bp, Artificial
  primer
cgggatccgcttctgtcagctttcagg

SEQ ID NO: 8 - DNA, 29 bp, Artificial
  primer
cccaagcttttaatttcctgtatcgaaga